(19) United States
(12) Patent Application Publication
(10) Pub. No.: US 2009/0025722 A1
(43) Pub. Date: Jan. 29, 2009

(54) ADAPTER FOR INHALATION APPLIANCES FOR TREATMENT OF ARTIFICIALLY VENTILATED PATIENTS

(76) Inventors: Michael Pieper, Biberach (DE); Herbert Hurtig, Biberach (DE); Matthaeus Mueller, Tannheim (DE); Hans Schmitt, Ingelheim (DE); Juergen Schraivogel, Ummendorf (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(21) Appl. No.: 12/162,712

(22) PCT Filed: Feb. 1, 2007
(86) PCT No.: PCT/EP07/50987

§ 371 Date: Sep. 29, 2008

(30) Foreign Application Priority Data
Feb. 1, 2006 (DE) 102006004528.9

(51) Int. Cl. A61M 15/00

(52) U.S. Cl. 128/203.15

(57) ABSTRACT

The invention relates to a device for delivery of respiratory gases in conjunction with a powder inhaler for inhaled administration of pharmaceuticals, pharmaceutical mixtures or pharmaceutical formulations to patients who are attached to respirators. The pharmaceuticals, pharmaceutical mixtures or pharmaceutical formulations are preferably administered to patients who are being artificially ventilated through a mask or via a tracheal tube under anesthesia.

### BACKGROUND TO THE INVENTION

The invention relates to a device for supplying respiratory gases in conjunction with a powder inhaler for the inhalative administration of pharmaceutical substances, pharmaceutical mixtures or pharmaceutical formulations to patients connected to ventilators. It is preferably used to administer pharmaceutical substances, pharmaceutical mixtures or pharmaceutical formulations to anaesthetised patients who are being ventilated through a mask or a tracheal tube.

### PRIOR ART

Ventilators supply the patient who is to be ventilated with respiratory gas, e.g. through supply tubes. Often there is a need to administer drugs to ventilated patients by the inhalative route. This is the case for example in patients with pulmonary diseases, e.g. Inflammatory or obstructive lung diseases such as asthma or COPD. In these patients, topical treatment of the lungs is often advantageous, in order to achieve a high enough topical concentration of active substance in the lungs and reduce systemic side effects of the drugs. However, it may also be necessary, or therapeutically useful, to administer drugs to the patient by inhalation for systemic effect.

As is known from US 04951661 or WO02/089887, a nebuliser can deliver an aerosol into the tube carrying the respiratory gas by means of a T-shaped member which is inserted into the supply tube of the ventilator. These nebulisers generate an aerosol by means of energy, e.g. by ultrasound or compressed air or compressed propellant gas. At the same time, an airstream is produced which carries the aerosol away from the nebuliser.

From WO04/098689 a nebuliser connecting device for ventilators is known which allows easier attachment and removal of a nebuliser without having to interrupt or negatively affect the supply of respiratory gas during the connecting or disconnecting of the nebuliser. The nebuliser connecting device is designed for the ventilator by the provision of special respiratory air supply means, connecting means and closure means.

The basis of the inhalative administration of powdered pharmaceutical formulations in capsules is the generation of an aerosol by the active inspiration of the patient without any further addition of propellant gas. The pressure difference produced by the patient breathing in causes an air flow into the lungs. If this air flow is guided through a mouthpiece of a powder capsule inhaler, the perforated medicament capsule in the inhaler is set vibrating. This process causes the capsule to be emptied, the aerosol to be generated and the medicament to be conveyed into the respiratory tract.

In the known method of connecting an inhaler to a ventilating system as described above, inspiration does not produce a pressure difference. The known method is thus not suitable for administering medicaments in powder form. This is particularly true of medicaments in powder form that are formulated in capsules for inhalation with powder inhalers such as, for example, the HandiHaler, Xcelovair, GyroHaler or Aerolizer. If these powder inhalers are connected to a ventilating system known from the prior art, this does not result in suitable generation and delivery of a medicament-containing aerosol.

In the journal MMW-Fortschritt Medizin No. 11, 2005, p. 44/192 ff, it also states that dry powder inhalers cannot be used in ventilation systems (cf. Table 2).

### BRIEF SUMMARY OF THE INVENTION

The basis of the present invention is to design an optimally constructed adapter or a separate device to allow pharmaceutical substances, pharmaceutical mixtures or pharmaceutical formulations to be administered by inhalation in conjunction with a powder inhaler through a ventilating system.

This aim is achieved by a device according to claim 1. Advantageous further features are recited in the subsidiary claims.

### DETAILED DESCRIPTION OF THE INVENTION

The present invention relates to a device for supplying respiratory gases in conjunction with a powder inhaler for the inhalative administration of pharmaceutical substances, pharmaceutical mixtures or pharmaceutical formulations to patients connected to ventilators. They are preferably used to administer pharmaceutical substances, pharmaceutical mixtures or pharmaceutical formulations to anaesthetised patients who are being ventilated through a mask or a tracheal tube. Pharmaceutical substances, pharmaceutical mixtures or pharmaceutical formulations in powder form, which are formulated in capsules for aerosol production and are used in powder inhalers (e.g. HandiHaler®, Xcelovair®, Aerolizer® or GyroHaler®), are preferred. Most particularly preferred are pharmaceutical substances, pharmaceutical mixtures or pharmaceutical formulations in powder form, which are formulated in capsules for aerosol production and are used in the HandiHaler®.

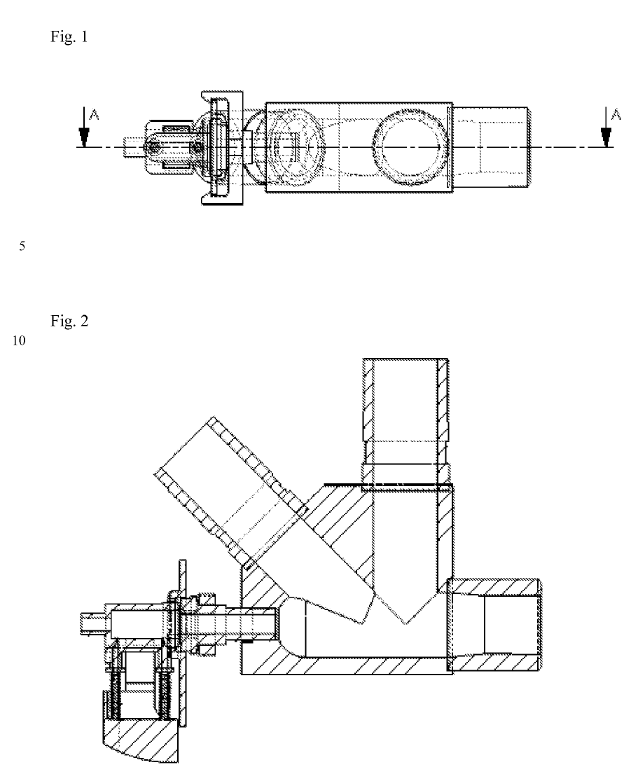

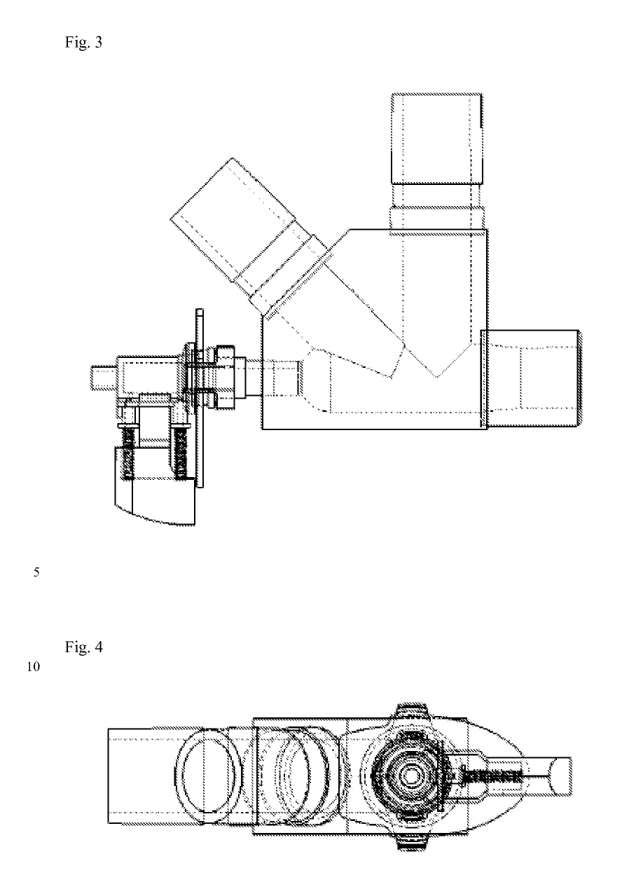

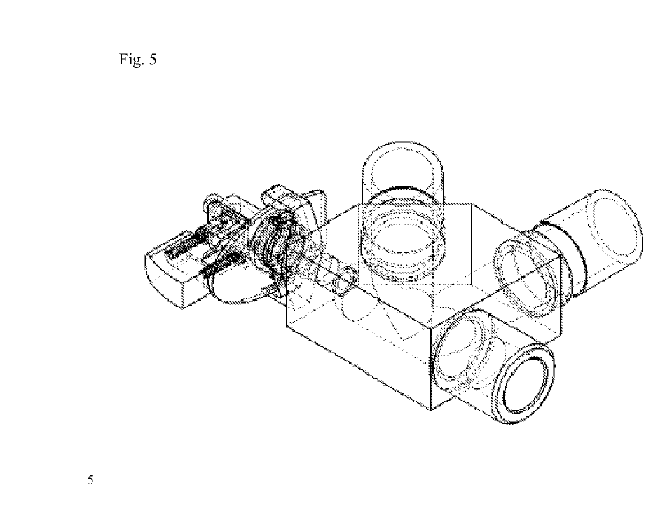

The problem is solved by the adapter according to the invention as shown in FIGS. 1-5.

According to the invention the inhaler is connected directly to the adapter for the ventilating system at a corresponding connector. Another possibility is to place the powder capsules without an inhaler directly in a corresponding chamber in the ventilating air-supplying tube of the ventilating system.

The ventilating air actively supplied under pressure by the ventilating system causes the capsule to vibrate and thus release the medicament and generate an aerosol. This aerosol is conveyed directly into the airways without any active inspiration by the patient.

The aerosol generated by the powder inhaler is thus taken in directly at the site of generation of the respiratory gas flow and supplied to the patient's airway. According to the invention the generation of the aerosol and its intake through the respiratory air-supplying gas current are advantageously located along the same axis as the tube that supplies the mixture of respiratory gas and aerosol to the patient. The aerosol is thus supplied to the patient with minimal impact in the respiratory air-supplying tube system. This ensures that the patient is advantageously supplied with active substances in aerosol form. Moreover, long-term inhalative medication or existing medication or administration of active substance to the patient which has already been carried out using the inhaler can be continued according to the invention even after the patient has been connected up to a ventilator.

The air current of the ventilating system can either be continuously supplied through the nebuliser connected to the adapter, or it can be supplied directly through a valve that is operated manually or automatically, circumventing the powder inhaler.

The powder inhaler is a device which is commercially obtainable, sold under the name HandiHaler®. It is also described in EP 0 703 800 B1 or EP 0 911 047 A1. The inhaler known from the above mentioned specifications has a dish-shaped lower part and an equally dish-shaped lid which fits it, these two parts being capable of being flipped apart for use, about a joint provided in the edge portion. Between the lower part and the lid, a mouthpiece which can also be flipped open and a plate below it with a capsule holder provided underneath also act on the joint. After the individual assemblies have been flipped open the patient can insert a drug-filled capsule in the capsule holder, pivot the plate and capsule holder and the mouthpiece into the lower part and pierce the capsule by means of a spring loaded actuating member projecting laterally from the lower part. The patient being treated then draws the pharmaceutical composition into his airway by sucking on the mouthpiece.

The pharmaceutically active substances, substance formulations or mixtures of substances used may be any inhalable compounds, such as e.g. inhalable macromolecules, as disclosed in EP 1 003 478. Preferably, substances, substance formulations or mixtures of substances which are taken by inhalation are used for treating respiratory complaints.

Particularly preferred in this context are pharmaceutical compositions selected from among the anticholinergics, betamimetics, steroids, phosphodiesterase IV inhibitors, LTD4-antagonists and EGFR-kinase inhibitors, antiallergics, ergot alkaloid derivatives, triptanes, CGRP antagonists, phosphodiesterase-V inhibitors, and combinations of active substances of this kind, e.g. betamimetics plus anticholinergics or betamimetics plus antiallergics. In the case of combinations at least one of the active substances contains chemically bound water. Anticholinergic-containing active substances are preferably used, as monopreparations or in the form of combined preparations.

The following are specific examples of the active ingredients or the salts thereof:

Anticholinergics to be used are preferably selected from among tiotropium bromide, oxitropium bromide, flutropium bromide, ipratropium bromide, glycopyrronium salts, trospium chloride, tolterodine, tropenol 2,2-diphenylpropionate methobromide, scopine 2,2-diphenylpropionate methobromide, scopine 2-fluoro-2,2-diphenylacetate-methobromide, tropenol 2-fluoro-2,2-diphenylacetate-methobromide, tropenol 3,3′,4,4′-tetrafluorobenzilate methobromide, scopine 3,3′,4,4′-tetrafluorobenzilate methobromide, tropenol 4,4′-difluorobenzilate methobromide, scopine 4,4′-difluorobenzilate methobromide, tropenol 3,3′-difluorobenzilate methobromide, scopine 3,3′-difluorobenzilate methobromide, tropenol 9-hydroxy-fluorene-9-carboxylate methobromide, tropenol 9-fluoro-fluorene-9-carboxylate methobromide, scopine 9-hydroxy-fluorene-9-carboxylate methobromide, scopine 9-fluoro-fluorene-9-carboxylate methobromide, tropenol 9-methyl-fluorene-9-carboxylate methobromide, scopine 9-methyl-fluorene-9-carboxylate methobromide, cyclopropyltropine benzilate methobromide, 2,2-diphenylpropionate cyclopropyltropine methobromide, cyclopropyltropine 9-hydroxy-xanthene-9-carboxylate methobromide, cyclopropyltropine benzilate methobromide, 2,2-diphenylpropionate cyclopropyltropine methobromide, cyclopropyltropine 9-hydroxy-xanthene-9-carboxylate methobromide, cyclopropyltropine 9-methyl-fluorene-9-carboxylate methobromide, cyclopropyltropine 9-methyl-xanthene-9-carboxylate methobromide, cyclopropyltropine 9-hydroxy-fluorene-9-carboxylate methobromide, methyl cyclopropyltropine 4,4′-difluorobenzilate methobromide, tropenol 9-hydroxy-xanthene-9-carboxylate methobromide, scopine 9-hydroxy-xanthene-9-carboxylate methobromide, tropenol 9-methyl-xanthene-9-carboxylate methobromide, scopine 9-methyl-xanthene-9-carboxylate methobromide, tropenol 9-ethyl-xanthene-9-carboxylate methobromide, tropenol 9-difluoromethyl-xanthene-9-carboxylate methobromide and scopine 9-hydroxymethyl-xanthene-9-carboxylate methobromide, optionally in the form of the racemates, enantiomers or diastereomers thereof and optionally in the form of the solvates and/or hydrates thereof.

Betamimetics which may be used are preferably selected from among albuterol, bambuterol, bitolterol, broxaterol, carbuterol, clenbuterol, fenoterol, formoterol, hexoprenaline, ibuterol, indacaterol, isoetharine, isoprenaline, levosalbutamol, mabuterol, meluadrine, metaproterenol, orciprenaline, pirbuterol, procaterol, reproterol, rimiterol, ritodrine, salmeterol, salmefamol, soterenot, sulphonterol, tiaramide, terbutaline, tolubuterol, CHF-1035, HOKU-81, KUL-1248, 3-(4-{6-[2-hydroxy-2-(4-hydroxy-3-hydroxymethyl-phenyl)-ethylamino]-hexyloxy}-butyl)-benzenesulphonamide, 5-[2-(5,6-diethyl-indan-2-ylamino)-1-hydroxy-ethyl]-8-hydroxy-1H-quinolin-2-one, 4-hydroxy-7-[2-{[2-{[3-(2-phenylethoxy)propyl]sulphonyl}ethyl]-amino}ethyl]-2(3H)-benzothiazolone, 1-(2-fluoro-4-hydroxyphenyl)-2-[4-(1-benzimidazolyl)-2-methyl-2-butylamino]ethanol, 1-[3-(4-methoxybenzyl-amino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, 5-hydroxy-8-(1-hydroxy-2-isopropylaminobutyl)-2H-1,4-benzoxazin-3-(4H)-one, 1-(4-amino-3-chloro-5-trifluoromethylphenyl)-2-tert.-butylamino)ethanol and 1-(4-ethoxycarbonylamino-3-cyano-5-fluorophenyl)-2-(tert.-butylamino)ethanol, optionally in the form of the racemates, enantiomers or diastereomers thereof and optionally in the form of the pharmacologically acceptable acid addition salts, solvates and/or hydrates thereof.

Steroids which may be used are preferably selected from among prednisolone, prednisone, butixocortpropionate, RPR-106541, flunisolide, beclomethasone, triamcinolone, budesonide, fluticasone, mometasone, ciclesonide, rofleponide, ST-126, dexamethasone, (S)-fluoromethyl 6α,9α-difluoro-17α-[(2-furanylcarbonyl)oxy]-11β-hydroxy-16α-methyl-3-oxo-androsta-1,4-diene-17β-carbothionate, (S)-(2-oxo-tetrahydro-furan-3 S-yl) 6α,9α-difluoro-11β-hydroxy-16α-methyl-3-oxo-17α-propionyloxy-androsta-1,4-diene-17β-carbothionate and etiprednol-dichloroacetate (BNP-166), optionally in the form of the racemates, enantiomers or diastereomers thereof and optionally in the form of the salts and derivatives thereof, the solvates and/or hydrates thereof. PDE IV inhibitors which may be used are preferably selected from among enprofyllin, theophyllin, roflumilast, ariflo (cilomilast), CP-325,366, BY343, D-4396 (Sch-351591), AWD-12-281 (GW-842470), N-(3,5-dichloro-1-oxo-pyridin-4-yl)-4-difluoromethoxy-3-cyclopropylmethoxybenzamide, NCS-613, pumafentine, (−)ₚ-[(4aR*,10bS*)-9-ethoxy-1,2,3,4,4a,10b-hexahydro-8-methoxy-2-methylbenzo[s][1,6]naphthyridin-6-yl]-N,N-diisopropylbenzamide, (R)-(+)-1-(4-bromobenzyl)-4-[(3-cyclopentyloxy)-4-methoxyphenyl]-2-pyrrolidone, 3-(cyclopentyloxy-4-methoxyphenyl)-1-(4-N′-[N-2-cyano-S-methyl-isothioureido]benzyl)-2-pyrrolidone, cis[4-cyano-4-(3-cyclopentyloxy-4-methoxyphenyl)cyclohexane-1-carboxylic acid], 2-carbomethoxy-4-cyano-4-(3-cyclopropylmethoxy-4-difluoromethoxyphenyl)cyclohexan-1-one, cis[4-cyano-4-(3-cyclopropylmethoxy-4-difluoromethoxyphenyl)cyclohexan-1-ol], (R)-(+)-ethyl[4-(3-cyclopentyloxy-4-methoxyphenyl)pyrrolidin-2-ylidene]acetate, (S)-(−)-ethyl[4-(3-cyclopentyloxy-4-methoxyphenyl)pyrrolidin-2-ylidene]acetate, CDP840, Bay-198004, D-4418, PD-168787, T-440, T-2585, arofyllin, atizoram, V-11294A, CI-1018, CDC-801, CDC-3052, D-22888, YM-58997, Z-15370, 9-cyclopentyl-5,6-dihydro-7-ethyl-3-(2-thienyl)-9H-pyrazolo[3,4-c]-1,2,4-triazolo[4,3-a]pyridine and 9-cyclopentyl-5,6-dihydro-7-ethyl-3-(tert-butyl)-9H-pyrazolo[3,4-c]-1,2,4-triazolo[4,3-a]pyridine, optionally in the form of the racemates, enantiomers or diastereomers thereof and optionally in the form of the pharmacologically acceptable acid addition salts, solvates and/or hydrates thereof. LTD4-antagonists which may be used are preferably selected from among montelukast, 1-(((R)-(3-(2-(6,7-difluoro-2-quinolinyl)ethenyl)phenyl)-3-(2-(2-hydroxy-2-propyl)phenyl)thio)methylcyclopropane-acetic acid, 1-(((1(R)-3 (3-(2-(2,3-dichlorothieno[3,2-b]pyridin-5-yl)-(E)-ethenyl)phenyl)-3-(2-(1-hydroxy-1-methylethyl)phenyl)propyl)thio)-methyl)cyclopropane-acetic acid, pranlukast, zafirlukast, [2-[[2-(4-tert-butyl-2-thiazolyl)-5-benzofuranyl]oxymethyl]phenyl]acetic acid, MCC-847 (ZD-3523), MN-001, MEN-91507 (LM-1507), VUF-5078, VUF-K-8707 and L-733321, optionally in the form of the racemates, enantiomers or diastereomers thereof, optionally in the form of the pharmacologically acceptable acid addition salts thereof and optionally in the form of the salts and derivatives thereof, the solvates and/or hydrates thereof.

EGFR-kinase inhibitors which may be used are preferably selected from among cetuximab, trastuzumab, ABX-EGF, Mab ICR-62, 4-[(3-chloro-4-fluorophenyl)amino]-6-{[4-(morpholin-4-yl)-1-oxo-2-buten-1-yl]amino}-7-cyclopropylmethoxy-quinazoline, 4-[(R)-(1-phenyl-ethyl)amino]-6-{[4-(morpholin-4-yl)-1-oxo-2-buten-1-yl]amino}-7-cyclopentyloxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline, 4-[(3-chloro-4-fluoro-phenyl)amino]-6-[2-((S)-6-methyl-2-oxo-morpholin-4-yl)-ethoxy]-7-methoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline, 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline, 4-[(3-ethynyl-phenyl)amino]-6,7-bis-(2-methoxy-ethoxy)-quinazoline, 4-[(R)-(1-phenyl-ethyl)amino]-6-(4-hydroxy-phenyl)-7H-pyrrolo[2,3-d]pyrimidine, 3-cyano-4-[(3-chloro-4-fluorophenyl)amino]-6-{[4-(N,N-dimethylamino)-1-oxo-2-buten-1-yl]amino}-7-ethoxy-quinoline, 4-[(R)-(1-phenyl-ethyl)amino]-6-{[4-((R)-6-methyl-2-oxo-morpholin-4-yl)-1-oxo-2-buten-1-yl]amino}-7-methoxy-quinazoline, 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline, 4-[(3-ethynyl-phenyl)amino]-6-{[4-(5,5-dimethyl-2-oxo-morpholin-4-yl)-1-oxo-2-buten-1-yl]amino}-quinazoline, 4-[(3-chloro-4-fluoro-phenyl)amino]-6-{2-[4-(2-oxo-morpholin-4-yl)-piperidin-1-yl]-ethoxy}-7-methoxy-quinazoline, 4-[(3-chloro-4-fluoro-phenyl)amino]-6-(trans-4-amino-cyclohexan-1-yloxy)-7-methoxy-quinazoline, 4-[(3-chloro-4-fluoro-phenyl)amino]-6-(trans-4-methanesulphonylamino-cyclohexan-1-yloxy)-7-methoxy-quinazoline, 4-[(3-chloro-4-fluoro-phenyl)amino]-6-(tetrahydropyran-3-yloxy)-7-methoxy-quinazoline, 4-[(3-chloro-4-fluoro-phenyl)amino]-6-{1-[(morpholin-4-yl)carbonyl]-piperidin-4-yloxy}-7-methoxy-quinazoline, 4-[(3-chloro-4-fluoro-phenyl)amino]-6-(piperidin-3-yloxy)-7-methoxy-quinazoline, 4-[(3-chloro-4-fluoro-phenyl)amino]-6-[1-(2-acetylamino-ethyl)-piperidin-4-yloxy]-7-methoxy-quinazoline, 4-[(3-chloro-4-fluoro-phenyl)amino]-6-(tetrahydropyran-4-yloxy)-7-ethoxy-quinazoline, 4-[(3-chloro-4-fluoro-phenyl)amino]-6-{trans-4-[(morpholin-4-yl)carbonylamino]-cyclohexan-1-yloxy}-7-methoxy-quinazoline, 4-[(3-chloro-4-fluoro-phenyl)amino]-6-{1-[(piperidin-1-yl)carbonyl]-piperidin-4-yloxy}-7-methoxy-quinazoline, 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-(trans-4-ethanesulphonylamino-cyclohexan-1-yloxy)-7-methoxy-quinazoline, 4-[(3-chloro-4-fluoro-phenyl)amino]-6-(1-methanesulphonyl-piperidin-4-yloxy)-7-(2-methoxy-ethoxy)-quinazoline, 4-[(3-chloro-4-fluoro-phenyl)amino]-6-[1-(2-methoxy-acetyl)-piperidin-4-yloxy]-7-(2-methoxy-ethoxy)-quinazoline, 4-[(3-ethynyl-phenyl)amino]-6-(tetrahydropyran-4-yloxy]-7-methoxy-quinazoline, 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline, 4-[(3-chloro-4-fluoro-phenyl)amino]-6-{cis-4-[(morpholin-4-yl)carbonylamino]-cyclohexan-1-yloxy}-7-methoxy-quinazoline, 4-[(3-chloro-4-fluoro-phenyl)amino]-6-{1-[2-(2-oxopyrrolidin-1-yl)ethyl]-piperidin-4-yloxy}-7-methoxy-quinazoline, 4-[(3-ethynyl-phenyl)amino]-6-(1-acetyl-piperidin-4-yloxy)-7-methoxy-quinazoline, 4-[(3-ethynyl-phenyl)amino]-6-(1-methyl-piperidin-4-yloxy)-7-methoxy-quinazoline, 4-[(3-ethynyl-phenyl)amino]-6-(1-methanesulphonyl-piperidin-4-yloxy)-7-methoxy-quinazoline, 4-[(3-chloro-4-fluoro-phenyl)amino]-6-(1-methyl-piperidin-4-yloxy)-7-(2-methoxy-ethoxy)-quinazoline, 4-[(3-ethynyl-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(N-methyl-N-2-methoxyethyl-amino)carbonyl]-piperidin-4-yloxy}-7-methoxy-quinazoline, 4-[(3-chloro-4-fluoro-phenyl)amino]-6-(1-ethyl-piperidin-4-yloxy)-7-methoxy-quinazoline, 4-[(3-chloro-4-fluoro-phenyl)amino]-6-[cis-4-(N-methanesulphonyl-N-methyl-amino)-cyclohexan-1-yloxy]-7-methoxy-quinazoline, 4-[(3-chloro-4-fluoro-phenyl)amino]-6-[cis-4-(N-acetyl-N-methyl-amino)-cyclohexan-1-yloxy]-7-methoxy-quinazoline, 4-[(3-chloro-4-fluoro-phenyl)amino]-6-(trans-4-methylamino-cyclohexan-1-yloxy)-7-methoxy-quinazoline, 4-[(3-chloro-4-fluoro-phenyl)amino]-6-[trans-4-(N-methanesulphonyl-N-methyl-amino)-cyclohexan-1-yloxy]-7-methoxy-quinazoline, 4-[(3-chloro-4-fluoro-phenyl)amino]-6-(trans-4-dimethylamino-cyclohexan-1-yloxy)-7-methoxy-quinazoline, 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline, 4-[(3-chloro-4-fluoro-phenyl)amino]-6-(1-methanesulphonyl-piperidin-4-yloxy)-7-methoxy-quinazoline, 4-[(3-chloro-4-fluoro-phenyl)amino]-6-(1-cyano-piperidin-4-yloxy)-7-methoxy-quinazoline, and 4-[(3-chloro-4-fluoro-phenyl)amino]-6-{1-[(2-methoxyethyl)carbonyl]-piperidin-4-yloxy}-7-methoxy-quinazoline, optionally in the form of the racemates, enantiomers or diastereomers thereof, optionally in the form of the pharmacologically acceptable acid addition salts thereof, the solvates and/or hydrates thereof.

By acid addition salts with pharmacologically acceptable acids which the compounds may be capable of forming are meant, for example, salts selected from among the hydrochloride, hydrobromide, hydriodide, hydrosulphate, hydrophosphate, hydromethanesulphonate, hydronitrate, hydromaleate, hydroacetate, hydrobenzoate, hydrocitrate, hydrofumarate, hydrotartrate, hydroxalate, hydrosuccinate, hydrobenzoate and hydro-p-toluenesulphonate, preferably the hydrochloride, hydrobromide, hydrosulphate, hydrophosphate, hydrofumarate and hydromethanesulphonate.

Examples of antiallergics are: disodium cromoglycate, nedocromil.

Examples of ergot alkaloids are: dihydroergotamine, ergotamine.

Examples of substances suitable for inhalation include medicaments, medicament formulations and mixtures containing the above-mentioned active substances, and the salts and esters thereof and combinations of these active substances, salts and esters.

1. Device for supplying respiratory gases in conjunction with powder inhalers for administering pharmaceutical substances, pharmaceutical mixtures or pharmaceutical formulations, characterised in that the device is an adapter according to FIGS. 1-5 or the powder capsule without an inhaler is placed directly in a corresponding chamber in the respiratory air-supplying tube of the ventilating system.

2. Device according to claim 1, characterised in that the pharmaceutical substance, pharmaceutical mixtures or pharmaceutical formulations is a pharmaceutically active substance for treating respiratory complaints.

3. Device according to claim 1, characterised in that the pharmaceutically active substance is a substance selected from among the following group of anticholinergics, betamimetics, steroids, phosphodiesterase IV inhibitors, LTD4-antagonists and EGFR-kinase inhibitors, antiallergics, ergot alkaloid derivatives, triptanes, CGRP antagonists, phosphodiesterase-V inhibitors.

4. Device according to claim 1, characterised in that the powder inhaler is a multi-dose device.

5. Device according to claim 1, characterised in that the powder inhaler is a single-dose device.

6. Device according to claim 5, characterised in that the powder inhaler is a single-dose device, wherein the pharmaceutical substance, pharmaceutical mixtures or pharmaceutical formulations is present in a capsule.

7. Use of the device according to claim 1 for administering a pharmaceutical substance, a pharmaceutical mixtures or pharmaceutical formulations to patients connected to a ventilator.

8. Use of the device according to claim 1 for administering a pharmaceutical substance, a pharmaceutical mixtures or pharmaceutical formulations to patients who are being ventilated through a mask or a tracheal tube.
